(19) United States
(12) Patent Application Publication
(10) Pub. No.: US 2017/0072004 A1
(43) Pub. Date: Mar. 16, 2017

(54) COMPOSITIONS AND METHODS FOR IMPROVING THE BIOAVAILABILITY OF CURCUMINOIDS

(71) Applicants: BRUCE HOWE, San Diego, CA (US); KODIMULE PRASAD, Bangalore (IN)

(72) Inventors: BRUCE HOWE, San Diego, CA (US); KODIMULE PRASAD, Bangalore (IN)

(21) Appl. No.: 14/850,514

(22) Filed: Sep. 10, 2015

(51) Int. Cl. A61K 36/9066; A61K 47/44; A23L 33/10; A23L 33/115; A23L 29/25; A61K 31/12; A23L 33/105

(52) U.S. Cl. CPC A61K 36/9066; A61K 31/12; A61K 47/44; A23L 33/105; A23L 33/115; A23L 29/25; A23L 33/10; A23V 2002/00

(57) ABSTRACT

Turmeric contains components such as curcuminoids which are beneficial to health and nutrition. Curcuminoids, including curcumin, are poorly absorbed by the body. The present invention provides compositions of curcuminoids having improved bioavailability. The present invention also provides methods of making such compositions and methods for their use in nutritional and therapeutic applications.

### BACKGROUND

Field of the Invention

The invention relates to compositions and methods for improving the bioavailability of curcumin. The invention further relates to use of such curcumin in the preparation of supplements and consumables and the administration of the inventive composition in nutritional and therapeutic applications.

Related Art

Curcuma longa, a perennial herb and member of the Zingiberaceae (ginger) family, grows to a height of three to five feet and is cultivated extensively in Asia, India, China, and other countries with a tropical climate. Dried Curcuma longa is the source of the spice turmeric, the ingredient that gives curry powder its characteristic yellow color. Turmeric is used extensively in foods for its flavor and color, as well as having a long tradition of use in the Chinese and Ayurvedic systems of medicine. Curcumin is a yellow color polyphenolic compound derived from turmeric. Its chemical name is bis-α,βunsaturated β-diketone and is commonly called diferuloylmethane. Commercial turmeric extract typically contains approximately 77% curcumin, 17% demethoxycurcumin, and 6% bisdemethoxycurcumin, and altogether are called curcuminoids. Turmeric has been shown in various animal models and human studies to be safe even used at very high doses. In spite of its efficacy and safety, turmeric has not yet been approved as a therapeutic agent. The major problem with the use of turmeric is related to the low bioavailability of its active component curcumin which poses significant pharmacological barriers for clinical application.

The active constituents of turmeric include the flavonoid curcumin (diferuloylmethane) and various volatile oils, including tumerone, atlantone, and zingiberone. Other constituents include sugars, proteins, and resins. Curcumin was characterized as an excellent molecule among many naturally occurring compounds for treatment and prevention of a wide variety of human diseases especially for cancer therapeutics. It was proven to be safe even used at very high doses (Jantarat, 2013). Curcumin is unstable at basic pH, and degrades within 30 min to trans-6-(40-hydroxy-30-methoxyphenyl)-2,4dioxo-5-hexanal, ferulic acid, feruloylmethane and vanillin (Lin et al., 2000). The absorption, metabolism and tissue distribution of curcumin has been studied in at least 10 studies performed in rodents over the past three decades. In an early study, a dose of 1 g/kg was administered to rats in the diet, about 75% of the dose was excreted in the feces and negligible amounts appeared in the urine (Wahlstrom & Blennow, 1978). The reasons for reduced bioavailability are low intrinsic activity, poor absorption, high rate of metabolism, inactivity of metabolic products, and rapid elimination and clearance from the body.

The solubility of curcumin in water is as low as 11 ng/mL. It is hydrolyzed rapidly in alkaline solutions and readily decomposed when exposed to bright light, high temperature or oxidative conditions (Jantarat, 2013).

In a study of oral curcumin (2 g/kg) in rats performed in Bangalore, India, the investigators suggested that co-administration of piperine may increase systemic bioavailability following oral dosing by as much as 154%, potentially by inhibition of xenobiotic glucuronidation (Shobha et al., 1998). As curcumin exhibits low oral bioavailability in rodents and may undergo intestinal metabolism, curcumin undergoes rapid first-pass metabolism and excretion in the bile.

What is needed in the art therefore are compositions and methods for increasing the bioavailability of curcumin and other curcuminoids in sources of curcumin such as turmeric extract.

### SUMMARY OF THE INVENTION

The inventors have surprisingly discovered that adding lecithin and optionally turmeric oil increases the bioavailability of curcumin such as that contained in turmeric extract. After adding these enhancers to turmeric extract, there was an improvement in the percentage of bioavailability of curcumin and longer retention time in the body as compared to conventional turmeric extract.

### BRIEF DESCRIPTION OF THE DRAWINGS

FIG. 1 shows the structure of curcumin.

FIG. 2 shows a diagram of non-limiting therapeutic uses of turmeric and curcumin.

FIG. 3 shows the concentration of curcumin in rat plasma after a single oral administration of turmeric extract and turmeric extract formulated with lecithin (1000 mg/kg body weight). All data showed a significant difference at P<0.01 versus the conventional turmeric extract group. Values are represented as a mean±SEMs (n=6).

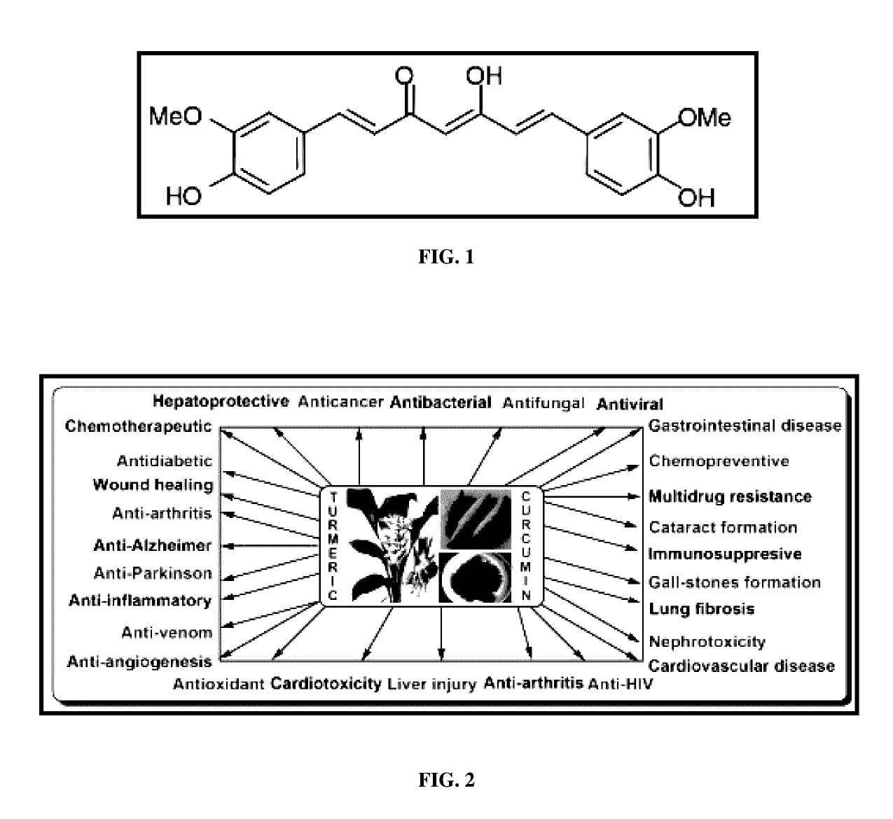

### DETAILED DESCRIPTION

The invention generally relates to methods and compositions for increasing the bioavailability of the bioactive constituents of turmeric including curcuminoids. As used herein, the term “turmeric” refers to material obtained from the herb curcuma longa. Turmeric for use with the invention includes, but is not limited to, raw turmeric, turmeric powders, turmeric oils, turmeric extracts, one or more purified curcuminoids, and combinations thereof.

As used herein, the phrase “increasing the bioavailability” refers to increasing the bioavailability of the referenced material relative to the bioavailability of the referenced material compared to a control set of conditions. In some aspects of the invention, the compositions and methods described herein increase the bioavailability of turmeric. The compositions of the invention may increase the bioavailability of one or more curcuminoids or a derivative thereof. The compositions and methods described herein may increase the bioavailability of turmeric extract or curcumin.

The term “bioavailability” as used herein refers to the degree and rate at which a substance is absorbed into a living system or is made available at the site of physiological activity.

As used herein, the term “curcuminoids” refers to curcumin, demethoxycurcumin, bisdemethoxycurcumin, and combinations thereof. In some aspects, the compositions and methods described herein increase the bioavailability of at least one curcuminoid or derivative thereof. In some aspects, the compositions and methods described herein increase the bioavailability of curcumin and/or derivative thereof.

The composition of the invention may be practiced with at least one purified curcuminoid or derivative thereof. As used herein, the term “purified” refers to a compound that has been separated from a component of the composition in which it was originally present. For example, a purified substance will comprise less than about 10%, preferably less than about 5%, and most preferably less than about 1% by weight of any other material. A purified compound may be 100% pure and be free of any other component.

The compositions and methods of the invention may be practiced with turmeric extract. Turmeric extract may contain about 95-98% of at least one curcuminoid or derivative thereof. Turmeric extract for use with the invention may contain curcuminoids in an amount of about 100%, 99% 98%, 97%, 96%, 95%, 94%, 93%, 92%, 91%, 90%, 80%, 70%, 60%, 50%, 40%, 30%, 20%, 10%, or any amount intervening these amounts. As used herein, uses of percentages to describe the relative amounts of components of the compositions described herein describe percentages of such components by their weight relative to the other components in the composition.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%, or any intervening range thereof.

In some aspects, the composition of the invention comprises turmeric extract, lecithin and turmeric oil. The composition can comprise turmeric extract, purified lecithin and turmeric oil. The composition can comprise turmeric extract, purified lecithin and purified turmeric oil. The composition can comprise turmeric extract, purified lecithin and optionally turmeric oil. The composition can comprise turmeric extract, purified lecithin and optionally purified turmeric oil. The amount of turmeric extract in the compositions of the invention can be, by weight, about 99%, 98%, 97%, 96%, 95%, 94%, 93%, 92%, 91%, 90%, 80%, 70%, 60%, 50%, or any amount intervening these amounts, with the remaining portion comprising lecithin (e.g. purified lecithin) and optionally turmeric oil (e.g. purified turmeric oil). In some aspects, the compositions of the invention are formulated, by weight, with about 1-2% lecithin (e.g. sunflower lecithin) and optionally 1-2% turmeric oil, with at least a portion of the remainder of the composition comprising turmeric extract. As noted herein, one or more of the components of the compositions described herein can be purified. For example, compositions of the invention may be formulated with turmeric extract, purified turmeric oil, and purified lecithin. The compositions of the invention may be formulated from one or more purified curcuminoids, lecithin (e.g. purified lecithin) and optionally turmeric oil. The amount of curcuminoids may be, by weight, about 99%, 98%, 97%, 96%, 95%, 94%, 93%, 92%, 91%, 90%, 80%, 70%, 60%, 50%, or any amount intervening these amounts, with the remaining portion comprising lecithin (e.g. purified lecithin) and optionally turmeric oil (e.g. purified turmeric oil).

The composition of the invention may be formulated to achieve increased bioavailability of turmeric by formulating the composition using particular ratios for the constituents of the compositions. The composition can comprise turmeric and lecithin in a ratio of between about 100:1, 90:1, 80:1, 70:1, 60:1, 50:1, 40:1, 30:1, 20:1, 10:1, 5:1 and 1:1. The composition can comprise turmeric extract and lecithin in a ratio of between about 100:1, 90:1, 80:1, 70:1, 60:1, 50:1, 40:1, 30:1, 20:1, 10:1, 5:1 and 1:1. The composition can comprise turmeric and purified lecithin in a ratio of between about 100:1, 90:1, 80:1, 70:1, 60:1, 50:1, 40:1, 30:1, 20:1, 10:1, 5:1 and 1:1. The composition can comprise turmeric extract and purified lecithin in a ratio of between about 100:1, 90:1, 80:1, 70:1, 60:1, 50:1, 40:1, 30:1, 20:1, 10:1, 5:1 and 1:1. The composition can comprise one or more purified curcuminoids or derivatives thereof and lecithin in a ratio of between about 100:1, 90:1, 80:1, 70:1, 60:1, 50:1, 40:1, 30:1, 20:1, 10:1, 5:1 and 1:1. The composition can comprise one or more purified curcuminoids or derivatives thereof and purified lecithin in a ratio of between about 100:1, 90:1, 80:1, 70:1, 60:1, 50:1, 40:1, 30:1, 20:1, 10:1, 5:1 and 1:1. The ratio of the constituents of the composition may similarly comprise any ratio intervening those described in the present disclosure.

The composition of the invention may comprise turmeric, lecithin and turmeric oil in a ratio suitable for increasing the bioavailability of the turmeric. The composition can comprise turmeric, lecithin and turmeric oil in a ratio of between about 100:1:1, 90:1:1, 80:1:1, 70:1:1, 60:1:1, 50:1:1, 40:1:1, 30:1:1, 20:1:1, 10:1:1, 5:1:1 and 1:1:1. The composition can comprise turmeric extract, lecithin and turmeric oil in a ratio of between about 100:1:1, 90:1:1, 80:1:1, 70:1:1, 60:1:1, 50:1:1, 40:1:1, 30:1:1, 20:1:1, 10:1:1, 5:1:1 and 1:1:1. The composition can comprise turmeric, purified lecithin and turmeric oil in a ratio of between about 100:1:1, 90:1:1, 80:1:1, 70:1:1, 60:1:1, 50:1:1, 40:1:1, 30:1:1, 20:1:1, 10:1:1, 5:1:1 and 1:1:1. The composition can comprise turmeric extract, purified lecithin and turmeric oil in a ratio of between about 100:1:1, 90:1:1, 80:1:1, 70:1:1, 60:1:1, 50:1:1, 40:1:1, 30:1:1, 20:1:1, 10:1:1, 5:1:1 and 1:1:1. The composition can comprise one or more purified curcuminoids or derivatives thereof, lecithin and turmeric oil in a ratio of between about 100:1:1, 90:1:1, 80:1:1, 70:1:1, 60:1:1, 50:1:1, 40:1:1, 30:1:1, 20:1:1, 10:1:1, 5:1:1 and 1:1:1. The composition can comprise one or more purified curcuminoids or derivatives thereof, purified lecithin and turmeric oil in a ratio of between about 100:1:1, 90:1:1, 80:1:1, 70:1:1, 60:1:1, 50:1:1, 40:1:1, 30:1:1, 20:1:1, 10:1:1, 5:1:1 and 1:1:1. The ratio of the constituents of the composition may similarly comprise any ratio intervening those described in the present disclosure. In addition, the composition may be formulated according to the ratios listed here, but wherein lecithin or turmeric oil is doubled or tripled relative to one another. For example, the composition may comprise turmeric, lecithin and turmeric oil in a ratio of 50:1:2, or 50:2:1.

The term “lecithin,” as used herein, refers to any or all of the phosphatides, pure or in blends comprising phosphatidylcholine, phosphatidylethanolamine, phosphatidylinositol, and/or phosphatidylserine, and/or other phosphatides regarded as lecithins. The term “lecithin” is intended to encompass any lecithin ingredient in any of the edible, commercially available forms.

Edible lecithins are well-known, widely available, and are described and defined in detail in the public literature. For example, they are described in: Kirk Othmer, Encyclopedia of Chemical Technology, Volume 14, pp. 250-269; in the Encyclopedia of Food Science, Peterson and Johnson, editors, Avi Publishing Co. 1978, pp. 461, 467; and LECITHINS, edited by Bernard F. Szuhaj, and Gary R. List, which was published by the American Oil Chemists' Society as a monograph. (Also see especially Chapter 8, Commercial Lecithin Products; Food Use of Soybean Lecithin, by W. E. Prosise.). The descriptions of all these references are incorporated by reference herein in their entirety and for all purposes. Lecithin for use with the invention may be sunflower lecithin.

In some aspects, the compositions and methods of the invention are practiced with an amount of lecithin sufficient to increase the bioavailability of turmeric. Such amounts of lecithin include any amount that is sufficient to increase the bioavailability of turmeric relative to a composition that lacks the amount of lecithin. In some aspects of the invention, such lecithin is in an amount sufficient to increase the bioavailability of one or more curcuminoids present in a turmeric extract. Some non-limiting embodiments of amounts of lecithin sufficient to increase the bioavailability of turmeric extract (e.g. one or more curcuminoids present in the turmeric extract) include, but are not limited to, less than about 10%, 9%, 8%, 7%, 6%, 5%, 4%, 3%, 2%, 1%, of the composition by weight, including any percentage intervening these listed percentages.

The composition of the invention may comprise at least one curcuminoid or derivative of such curcuminoids. The term “derivative” as used herein refers to a substance (e.g. curcuminoid) which comprises the same basic carbon skeleton and functionality as the parent compound, but can also bear one or more substituents or substitutions of the parent compound. Curcuminoid derivatives may be so modified to achieve, for example, greater efficacy and/or greater bioabsorption. Examples of curcuminoid derivatives for use with the invention, include but are not limited to, those disclosed in the references, the disclosures of which are incorporated herein by reference in their entirety for all purposes: US 20130190256; U.S. Pat. No. 8,962,674; U.S. Pat. No. 8,609,723, U.S. Pat. No. 8,609,723; EP 2436673; EP 2123637; EP 2436673; US 2006/0276536; WO 2012076696; WO 2014071438; CN 101570512; CN 104042569; WO 2008045534; US 2013/0296527; and EP 2768797.

In some aspects of the invention, one or more of the constituents of the composition are organic. As used herein, the term “organic” refers to, relating to, yielding, or involving the use of food produced with the use of feed or fertilizer of plant or animal origin without employment of chemically formulated fertilizers, growth stimulants, antibiotics, or pesticides. In some aspects, organic constituents for use with the composition of the invention are certified by an organization that is approved and recognized by the USDA National Organics Program.

In some aspects, the invention provides formulations of turmeric for administration to a subject, such as a human subject. Such formulations may assume any form suitable for oral administration to a subject including, but not limited to pills, capsules, tablets, liquids (e.g. beverages, or drops), gummies, pastes, emulsions, drops, syrups, gels, softgels, powders, or food supplements. The invention also contemplates formulating the compositions as cosmeceuticals, including, but not limited to, creams, gels, powders, milks, emulsions, liquids, sprays, foams, sticks, and pastes. The compositions of the invention may be administered through any suitable means, including topically, orally, sublingually, intra-ocularly, vaginally, intravenously, intramuscularly, intra-arterially or by suppository. Thus, the compositions of the invention may be formulated with a suitable pharmaceutical carrier for administering according to any of the preceding routes of administration. Compositions of the invention can comprise one or more carriers including, but not limited to, sodium citrate, dicalcium phosphate, fillers or extenders (such as starches, lactose, sucrose, glucose, mannitol, and silicic acid), binders (such as, for example, carboxymethyl-cellulose, alginates, gelatin, polyvinylpyrrolidinone, sucrose, and acacia), disintegrating agents (such as agar-agar, calcium carbonate, potato or tapioca starch, alginic acid, silicates, and sodium carbonate), buffering agents and combinations thereof.

The compositions of the invention can comprise one or more agents for improving the palatability of the composition. For example, the composition may comprise at least one sweetener, aromatic compound, flavoring, or a combination thereof. Similarly, the compositions of the invention may comprise agents to increase the antioxidant and/or nutritional value of the compositions. Such agents include, but are not limited to, vitamins, minerals, proteins, amino acids, and carbohydrates.

In some aspects, the invention comprises a method for treating a disease or disorder in a subject comprising administering to the subject a composition as disclosed herein. Such disorders and diseases include any disorder or disease capable of being treated with turmeric including, but not limited to, cataract formation, diabetes, wound healing, arthritis, Alzheimer's disease, Parkinson's disease, HIV, cardiovascular disease, liver injury, nephrotoxicity, lung fibrosis, gall stones, multidrug resistance and gastrointestinal disease. The compositions of the invention may also be administered as a hepatoprotective agent, anticancer agent, antibacterial agent, antifungal agent, antiviral agent, chemopreventative agent, and immunosuppressive agent. In still other embodiments, the compositions of the invention are administered as a general nutritional supplement. As used herein, the term “subject” refers to a mammal, including but not limited to humans, non-human primates, cattle, sheep, dogs, cats, rats, mice, horses, goats, pigs or poultry (e.g. chickens, ducks, and geese).

Particular embodiments of the present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

### Example 1

### Formulation of Turmeric Extract with Lecithin

Curcumin (98% purity) was procured from Sigma Aldrich, USA, (Lot #021M2144V). Chemicals required for the study included tetrahydrofuran, citric acid, double-distilled were of HPLC grade (Merck, Mumbai, India). Olive oil (Bilginaglu, 100% pure) was procured from Marbi A. S, Turkey- (Lot no 2-16).

Organic turmeric extract was admixed with organic lecithin (1% w/w) and organic turmeric oil (1% w/w). The mixed powder was transferred into dry plastic container and tightly sealed. The final formulation of the turmeric extract with enhanced bioavailability was: organic turmeric extract (98%); organic lecithin (1%); and organic turmeric oil (1%).

### Example 2

### Turmeric Extract Formulated with Lecithin has Increased Bioavailability

### Experimental Animals

Female Wistar rats weighing 250 g were used in this study in accordance with institutional guidelines and approval of local ethics authorities. The animals were fed with commercial pellet diet (Biogen, Bangalore, India) and water ad libitum. The animals were acclimatized to laboratory hygienic conditions for 10 days before starting the experiment. The animals were maintained in groups of six and were fasted for 8 h prior to the commencement of the study.

### Animal Treatment

Female Wistar albino rats were kept under a twelve-hour light/dark cycle on standard lab chow. Animals were fasted overnight and received organic turmeric extract and formulated organic turmeric extract from Example 1 at 1000 mg/kg body weight by oral gavage in olive oil. At 30, 60, 120, 180, 240 and 360 min, animals were exsanguinated under terminal anaesthesia. Group size was 6 rats per time point. Whole blood was collected by retro orbital plexus technique from rats into heparinized tubes, centrifuged immediately at 3000 rpm for 15 min; plasma was then decanted and stored at −80° C. until analysis.

### Sample Preparation

Curcumin were extracted from plasma by solid phase extraction. An aliquot of 50 μL plasma samples were transferred into a centrifuge tube, to which 100 μL of acetonitrile was added. Subsequently, the mixture was centrifuged at 10,000 rpm for 10 min following vortex mixing for 90 s. The organic supernatant was transferred into a clean centrifuge tube was injected onto the HPLC system.

### HPLC Analysis of Curcumin

A validated sensitive and selective high-performance liquid chromatography (HPLC) method using PDA detection was used for the determination and quantification of curcumin and its metabolites. The HPLC system consisted of a Shimadzu LC 20 AT equipped with a solvent delivery pump, a Rheodyne injector valve and a PDA (code) detector. The column used was reversed phase C18 analytical column (4.6×250 mm, particle size 5 μm), with mobile phase consisting of two components: A, tetrahydrofuran; B, citric acid (1%) (40:60) with isocratic gradient system at run time of 35 min. The flow rate was maintained at 1 mL/min at 25±2° C. The eluate was monitored at 420 nm. Retention time for curcumin, demethoxycurcumin, and bisdemethoxycurcumin were 18.52, 21.78 and 25.46 min respectively. Free curcumin is completely insoluble in water therefore the concentration of curcumin was calculated using standard curve of curcumin in ethanol.

### Pharmacokinetic Analysis

Pharmacokinetic calculations were performed on each individual set of data using the WinNonlin Standard Edition Version 2.1 by non-compartmental method. Pharmacokinetic results are represented as mean±SEM. Statistical analysis was performed by t test to compare different groups. The level of significance was set at p<0.05.

### Results and Discussion

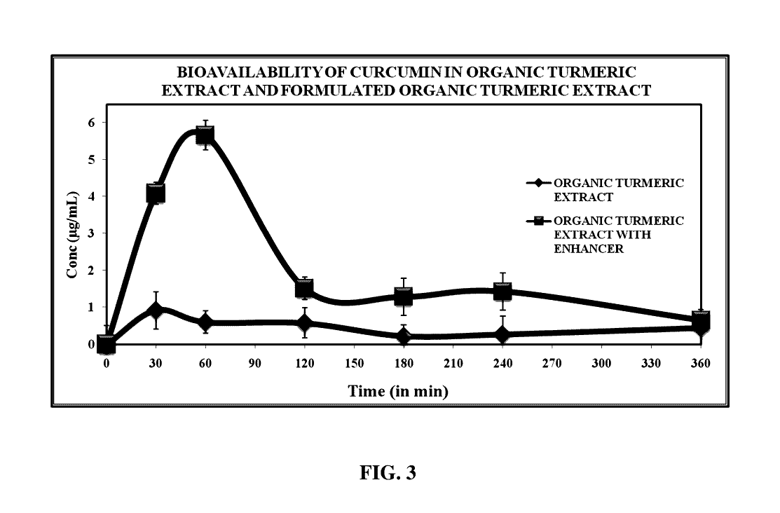

In the present investigation, organic turmeric extract and formulated organic turmeric extract was chosen for pharmacokinetics studies. FIG. 3 shows the mean plasma curcumin concentration in organic turmeric extract versus time profiles before and after oral administration of formulated organic turmeric extract at a dose of 1000 mg/kg body weight for each treatment group. The peak concentration (Cₘₐₓ) and time of peak concentration (Tₘₐₓ) were obtained directly from the individual plasma curcumin concentration versus time profiles. It was observed that at 30 min maximum curcumin concentration was detected in conventional turmeric extract (0.91±0.03 μg/mL) and was detected in 1 hr with formulated organic turmeric extract (5.66±0.61 μg/mL). However there was an improvement in percentage bioavailability of the formulated organic turmeric extract. Hence, organic turmeric extract with bioenhancer showed better absorption into blood with longer retention time as compared to conventional turmeric extract.

TABLE 1
Pharmacokinetic parameters derived from rat plasma

| Sample | Cₘₐₓ (μg/mL) | Tₘₐₓ (min) |
|---|---|---|
| Organic Turmeric extract | 0.91 ± 0.03 | 30 |
| Formulated Organic Turmeric extract | 5.66 ± 0.61 | 120 |

* Cₘₐₓ: maximum concentration; and Tₘₐₓ: time to reach Cmax

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1. Lin J. K., Pan M. H., Shiau S. Y. L. (2000) Recent studies on the biofunctions and biotransformations of curcumin. Biofactors, 13, 153-158.
2. Wahlstrom B., Blennow G. (1978) A study on the fate of curcumin in the rat. Acta Pharmacol Toxicol, 43, 86-92.
3. Ravindranath V, Chandrasekhara N. (1980) Absorption and tissue distribution of curcumin in rats. Toxicol, 16, 259-265.
4. Ireson C, Orr S, Jones D J L, et al. (2001) Characterization of metabolites of the chemopreventive agent curcumin in humans and rat hepatocytes and in the rat in vivo, and evaluation of their ability to inhibit phorbol ester-induced prostaglandin E production. Cancer Res, 61, 1058-1064.
5. Sharma R. A., Ireson C. R., Verschoyle R. D., et al. (2001) Effects of dietary curcumin on glutathione S-transferase and malondialdehyde-DNA adducts in rat liver and colon mucosa: relationship with drug levels. Clin Cancer Res, 7, 1452-1458.
6. Shoba G., Joy D., Joseph T., et al. (1998) Influence of piperine on the pharmacokinetics of curcumin in animals and human volunteers. Planta Med, 64, 353-356.
7. Dobelis I N (1986), ed. Magic and Medicine of Plants. Pleasantville, N.Y.: Reader's Digest Association, Inc.
8. Leung A. (1980) Encyclopedia of Common Natural Ingredients Used in Food, Drugs, and Cosmetics. New York, N.Y.: John Wiley: 313-314.
9. Khanna, M. M. (1999). Turmeric—Nature's precious gift. Current Sci. 76(10): 1351-1356.
10. Antony B. (2010) Composition to enhance the publication classification bioavailability of curcumin US 2010/0217045 A1.
11. Maiti, K.; Mukherjee, K.; Gantait, A.; Saha, B. P.; Mukherjee, P. K. (2007) Curcumin-phospholipid complex: Preparation, therapeutic evaluation and pharmacokinetic study in rats. Int. J. Pharm., 330, 155-163.

1. A composition comprising:
at least one curcuminoid or derivative thereof; and
lecithin;
wherein the at least one curcuminoid or derivative thereof and the lecithin are in a ratio sufficient to increase the bioavailability of the at least one curcuminoid or derivative thereof in a subject.

2. The composition of claim 1, wherein the at least one curcuminoid or derivative thereof and the lecithin are in a ratio of about 20:1 to about 100:1 by weight.

3. The composition of claim 1, wherein the at least one curcuminoid or derivative thereof and the lecithin are in a ratio of about 50:1 by weight.

4. The composition of claim 1, wherein the composition comprises about 95-98% the at least one curcuminoid or derivative thereof and about 2-5% of the lecithin by weight.

5. The composition of claim 1, wherein the composition comprises about 98% of the at least one curcuminoid or derivative thereof and about 2% of the lecithin by weight.

6. The composition of claim 1, wherein the composition comprises about 95-98% of the at least one curcuminoid or derivative thereof by weight.

7. The composition of claim 1, wherein the composition comprises about 98% of the at least one curcuminoid or derivative thereof by weight.

8. The composition of claim 1, wherein one or more of the at least one curcuminoid and the lecithin are purified.

9. The composition of claim 1, wherein the at least one curcuminoid or derivative thereof comprises at least one of curcumin, demethoxycurcumin, bisdemethoxycurcumin, or a derivative thereof.

10. The composition of claim 1, wherein the at least one curcuminoid or derivative thereof comprises turmeric extract.

11. The composition of claim 10, wherein the turmeric extract comprises about 95% to about 98% of the at least one curcuminoid or derivative thereof by weight.

12. The composition of claim 10, wherein the turmeric extract comprises about 98% of the at least curcuminoid or derivative thereof by weight.

13. The composition of claim 1, wherein the lecithin is sunflower lecithin.

14. The composition of claim 1, further comprising turmeric oil.

15. The composition of claim 14, wherein the at least one curcuminoid or derivative thereof, the lecithin and the turmeric oil are present in a ratio of about 100:1:1 by weight.

16. The composition of claim 14, wherein the composition comprises about 98% of the at least one curcuminoid or derivative thereof, about 1% lecithin, and about 1% turmeric oil by weight.

17. The composition of claim 1, further comprising acacia gum.

18. The composition of claim 1, wherein the composition is formulated as a pill, capsule, liquid, tablet, gummy, drops, beverage, powder, food supplement, gel or cosmeceutical.

19. A method of making a curcuminoid composition having increased bioavailability, the method comprising:
providing at least one curcuminoid or derivative thereof;
providing an amount of an amount of lecithin sufficient to increase the bioavailability of the at least one curcuminoid or derivative thereof in a subject relative to the at least one curcuminoid or derivative thereof in the absence of lecithin; and
combining the at least one curcuminoid or derivative thereof and the lecithin; and
optionally combining the at least one curcuminoid or derivative thereof and the lecithin with turmeric oil;
thereby providing a curcuminoid composition having increased bioavailability.

20. The method of claim 19, wherein the at least one curcuminoid or derivative thereof and the lecithin are combined in a ratio of about 20:1 to about 100:1 by weight.

21-37. (canceled)
